(19) United States
(12) Patent Application Publication
(10) Pub. No.: US 2008/0213362 A1
(43) Pub. Date: Sep. 4, 2008

(54) COMPOSITIONS AND METHODS USING TORSEMIDE

(76) Inventor: Lawrence Solomon, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 12/025,648

(22) Filed: Feb. 4, 2008

Related U.S. Application Data
(60) Provisional application No. 60/887,919, filed on Feb. 2, 2007.

(51) Int. Cl. A61K 9/44; A61K 31/44; A61K 31/4439; A61P 9/00; A61P 3/10; A61K 9/20

(52) U.S. Cl. 424/467; 514/349; 424/464; 514/342

(57) ABSTRACT

Dosage forms containing torsemide as the active ingredient, preferably provided at a relatively low dose or strength, are disclosed. The dosage forms can, be formulated as a tablet having a novel scoring pattern enabling division of the tablet to provide unique doses and dosing regimens to be administered to a patient in need of treatment or prevention using torsemide.

### CROSS-REFERENCE TO RELATED APPLICATION

This is a continuation of US Provisional Patent Application Ser. No. 60/887,919, filed Feb. 2, 2007.

### BACKGROUND

It is known to utilize torsemide, also known as torasemide, for its diuretic activity in the treatment of edema and congestive heart failure. It is also known to utilize torsemide to treat hypertension. The lowest strength in a marketed dose of torsemide is a 5 mg tablet, which is scored.

Studies have been performed that indicate that administering 2.5 mg of torsemide once daily has statistically significant anti-hypertensive effects. No published studies are available that describe utilizing doses below 2.5 mg daily. No teaching has been located that advises utilizing either a daily dose or a single dose of torsemide below 2.5 mg. It has been suggested that the 2.5 mg once daily dose of torsemide may have an antihypertensive effect that exceeds 50% of said effect of the 5 mg once daily dose.

Torsemide has been marketed as bisected scored tablets, which misleadingly suggests that the tablet may be broken into two portions and provide two equal half-doses. Torsemide is not known, to the best of the inventor's knowledge, to be available as tablets that are scored differently than the conventional bisection, or to provide more than two portions when broken along the score line(s), e.g., provided as a tablet, having a trisected, quadrisected or “pentasected” scoring pattern.

### BRIEF DESCRIPTION OF THE INVENTION

The invention concerns novel dosage forms comprising torsemide as the active ingredient in the dosage form. The active ingredient torsemide, is preferably, provided at a low strength as compared to known dosage forms, and can be formulated as a tablet having a novel scoring pattern. The novel scoring pattern can enable division of the tablet to provide unique doses and dosing regimens to be administered to a patient in need of treatment or prevention using torsemide. Compositions of the subject invention further include compressed tablets comprising two or more segments wherein at least one segment comprises a composition that is substantially free of an active pharmaceutical ingredient.

Another embodiment of the subject invention includes a dosage form comprising torsemide as a first active pharmaceutical ingredient, and a second active pharmaceutical ingredient that is different than torsemide. The second active pharmaceutical ingredient can be a known cardiovascular drug or can be an antidiabetic. A preferred antidiabetic is a thiozolidinedione, such as pioglitazone, rosiglitazone, or the like.

Methods of use of the subject invention include administering torsemide using a dose which is lower than previously known used; a treatment method or dosing regimen using a tablet, or portion thereof, containing a low dose of torsemide; and treating a patient with low-dose torsemide using a tablet having a novel scoring pattern, are also part of the subject invention.

These low-dose methods of use, as well as methods of using a torsemide tablet which has a novel scoring pattern, may also be beneficially administered to a patient suffering from diabetes or other disease or condition involving a deficiency or impairment of glucose metabolism, including treating a patient with metabolic syndrome.

### BRIEF DESCRIPTION OF THE DRAWINGS

FIG. 1 depicts a tablet dosage form comprising about 2 mg or less of torsemide, the tablet comprising a bisecting score.

FIG. 2 depicts a tablet dosage form comprising, torsemide, the tablet comprising two parallel score lines trisecting the tablet into three substantially equal portions.

FIG. 3 depicts a tablet dosage form comprising torsemide, the tablet comprising three parallel score lines quadrisecting the tablet into four substantially equal portions.

FIG. 4 depicts a tablet dosage form comprising torsemide, the tablet comprising four parallel score lines pentasecting the tablet into five substantially equal portions.

FIG. 5 depicts a tablet dosage form comprising torsemide, the tablet comprising intersecting score lines trisecting the tablet into three substantially equal portions.

FIG. 6 depicts a tablet dosage form comprising torsemide, the tablet comprising intersecting score lines quadrisecting the tablet into four substantially equal portions.

FIG. 7 depicts a tablet dosage form comprising torsemide, the tablet comprising intersecting score lines pentasecting the tablet into five substantially equal portions.

FIG. 8 depicts a tablet dosage form comprising torsemide, the tablet being configured as a layered dosage form leaving a substantially inactive middle segment.

FIG. 9 depicts a tablet dosage form as shown in FIG. 8f wherein the tablet includes a score formed in the middle segment.

FIG. 10 depicts a tablet dosage form comprising torsemide, the tablet being configured as a layered tablet having unitary segments.

FIG. 11 depicts a tablet dosage form comprising torsemide, configured as a layered tablet having a substantially inactive segment and a contiguous deeply scored active segment.

### DETAILED DESCRIPTION OF THE INVENTION

The invention concerns a novel dosage form comprising torsemide as an active pharmaceutical ingredient (API) in the dosage form. Torsemide is preferably provided in the dosage form at a strength which is lower than provided in previously known torsemide dosage forms. The lowest known strength of a marketed torsemide product is a tablet containing 5 mg of torsemide. Preferable embodiments of dosage forms of the subject invention contain torsemide at a strength of about 2 mg or less. The subject invention further includes a low-dose torsemide tablet provided with a score, such as a bisecting score, or a novel scoring pattern such as a trisecting, quadrisecting or pentasecting score to enable unique dosing regimens for torsemide.

Tablets of the subject invention are formulated to include, in addition to torsemide as the API, pharmaceutically acceptable excipients that are well known in the art. These excipients are formulated with the API into dosage forms, e.g., tablets, using known pharmaceutical formulation and manufacturing procedures as described in, for example, Remington's Pharmaceutical Sciences 20th Ed., Mack Publishing Co., Easton, Pa. (2000), Chapter 45, which is incorporated by reference.

In a further embodiment of the subject invention, the dosage form can include torsemide in combination with a second active pharmaceutical ingredient. In one preferred embodiment, the second API is another antihypertensive drug or a diuretic other than torsemide. More preferably, the second API is a diuretic other than a loop diuretic. Alternatively, the second API can be a drug having anti-diabetic properties. A preferred anti-diabetic drug used in combination with torsemide in a dosage form of the subject invention is a thiazolidinedione, which is also known as a “glitazone.” A preferred glitazone can be pioglitazone or rosiglitazone.

In addition, dosage forms of the subject invention can be formulated as layered tablets using accurately breakable tablet technologies as disclosed in Int'l Applications WO 2005/112,870; WO 2005/112,897; WO 2005/112,898; WO 2005/112,900; WO 2006/038,916; and US 2006/0003000, which are also incorporated herein by reference.

Novel methods of using torsemide including a method of treating a patient using a low-dose of torsemide, a treatment method using a tablet, or portion thereof, containing a low dose of torsemide, or treating a patient using a torsemide-containing tablet having a novel scoring pattern, are also disclosed as part of the subject invention. These methods of use that employ a low-dose torsemide tablet, and preferably using such tablet provided with a novel scoring pattern, may also be beneficial in a method of treating a patient suffering from diabetes, or, other glucose metabolism impairment, including patients with metabolic syndrome.

A dosage form comprising torsemide in accordance with the subject invention can provide a benefit when administered to a patient in that levels of blood sugar are not worsened. Torsemide administered to a diabetic patient or a pre-diabetic patient has been known to worsen blood sugar levels, even if administered during treatment with an anti-diabetic agent. The subject invention can advantageously provide antihypertensive or diuretic treatment to a patient without worsening blood sugar levels. This can be advantageous in treatment of a diabetic patient or a pre-diabetic patient.

The subject invention includes a method of treating a pre-diabetic patient or a diabetic patient with torsemide in a dosage form as described herein, without worsening blood sugar levels. The subject method of treatment can employ a dosage form comprising torsemide and an anti-diabetic agent in combination. In a preferred embodiment, the anti-diabetic component in the dosage form is a thiazolidinedione, (glitazone), ergs pioglitazone or rosiglitazone.

In addition, administration of a 2 mg or less once daily dose of torsemide can have antihypertensive efficacy, which has not been previously disclosed or suggested in the medical or pharmaceutical literature. The subject invention can provide unexpected benefits for torsemide administered at doses below 2.5 mg once daily. For example, administration to a patient of a dosage form containing a 2 mg once daily dose of torsemide can unexpectedly retain greater than 40% of the antihypertensive benefits of the 5 mg once daily dose. Administration to a patient of a dosage form containing a 1 mg once daily dose of torsemide can unexpectedly retain greater than 20% of the antihypertensive benefits of the 5 mg once daily dose. Administration to a patient of a dosage form containing a 2 mg 1.5 once daily dose of torsemide can unexpectedly retain greater than 80% of the antihypertensive benefits of the 2.5 mg once daily dose, and administration to a patient of a dosage form containing a 1 mg once daily dose of torsemide can unexpectedly retain greater than 40% of the antihypertensive benefits of the 2.5 mg once daily dose.

The subject invention further includes dosage forms containing torsemide at known strengths, but comprising novel scoring patterns. For example, a trisected, quadrisected or pentasected tablet containing between about 5 to about 100 mg of torsemide, including a known tablet strength of 5, 10, 20, 50 or 1.00 mg, is part of the subject invention.

It has not been suggested to place a score into a tablet comprising torsemide when such tablet contains less than 5 mg of torsemide. It has also not been suggested to have more than a single bisecting score in any tablet comprising torsemide. It has further not been suggested that a scoring pattern of a tablet containing torsemide be created to delineate a segment of the tablet that contains less than 2.5 mg of torsemide. Specific scoring patterns are disclosed herein and are novel for a dosage form containing torsemide as the API. These scoring patterns include, but are not limited to, tablets having two parallel scores that delineate equal thirds of the amount of torsemide contained in the tablet, three parallel scores or a crossing score (e.g., two intersecting lines substantially perpendicular to one another) that delineate equal quarters of the dose, or a scoring pattern such as four parallel cores that delineate equal fifths of the whole dose. It would be apparent to a person of ordinary skill in the art that a plurality of “score lines” can be formed by a single compression step using an embossed tablet die or tablet punch where the embossing is formed as a particular pattern which corresponds to the resulting pattern formed in the tablet face. Accordingly, reference herein to a plurality of “score lines” includes a single score, a single score having a plurality of parallel lines formed at one compression step, or a score having a plurality of intersecting branches projecting from one or more points of intersection.

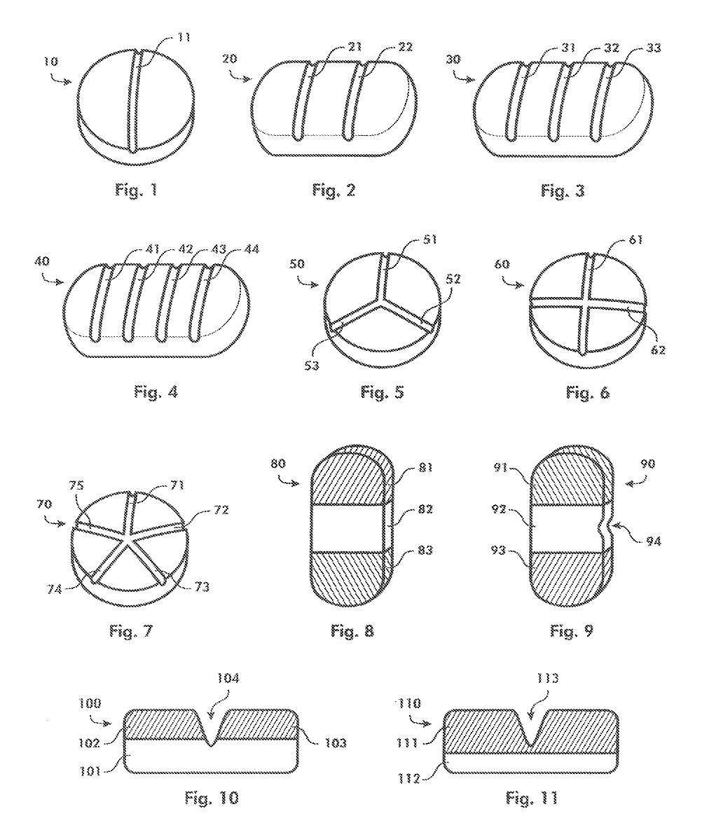

Examples of the unique scoring patterns for a torsemide containing dosage form are illustrated in the accompanying drawings. FIG. 1 depicts a compressed tablet 10 comprising a substantially homogeneous composition comprising torsemide, preferably about 2 mg or less of torsemide, as an API in the composition Tablet 10 comprises a bisecting score 11 in accordance with one embodiment of the subject invention.

Torsemide-containing tablets comprising parallel score lines which trisect, quadrisect, or pentasect the tablet are shown in FIGS. 2-4, respectively. Specifically, FIG. 2 depicts a capsule-shaped compressed torsemide tablet 20 comprising two parallel scores 21 and 22 formed in the same face of a tablet, perpendicular to the long axis of the tablet, and indicating divisions of the tablet into three substantially equal portions; divisions of a torsemide-containing tablet into four substantially equal portions can be provided by three parallel scores 31, 32, and 33, formed perpendicular to the long axis of a capsule-shaped tablet 30, as illustrated in FIG. 3; and FIG. 4 shows a capsule-shaped torsemide-containing tablet 40 having four parallel scores 41, 42, 43, and 44, which are formed in a face of the tablet to indicate five substantially equal divisions of the tablet.

Intersecting score lines can also be formed in the tablets to provide a torsemide-containing dosage form comprising a novel score pattern according to the subject invention. For example FIG. 5 depicts a conventional round, tablet 50 comprising torsemide but wherein the round tablet comprises intersecting score lines 51, 52, and 53 which meet in the approximate center point of a face of the tablet, trisecting the tablet into substantially equal thirds of the tablet. In FIG. 6, intersecting score lines 61 and 62, which form a “cross” pattern, quadrisecting torsemide tablet 60 into substantially four equal tablet portions are depicted. FIG. 7 shows a torsemide tablet 70 comprising intersecting score lines 71, 72, 73, 74, and 75, which pentasect the tablet into five substantially equal tablet portions.

FIGS. 8-11 illustrate novel, segmented tablet configurations as described in published patent applications WO 2005/112,870; WO 2005/112,897; WO 2005/12,898; WO 2005/112,900; WO 2006/038,916; and US 2006/0003000, incorporated by reference herein. FIG. 8 depicts a segmented tablet dosage form 80 comprising torsemide, the tablet being configured as a layered dosage form having a three segments 81, 82, and 83 wherein segments 81 and 83 (shaded) are formed from compositions comprising an API, preferably torsemide in amounts of about 2 mg or less in each segment. Middle segment 82 is formed by one or more layers of a substantially inactive composition, i.e., it is substantially free of any API, and forms a segment through which the tablet can be easily broken without breaking any of the active segment 81 or active segment 83. FIG. 9 depicts a tablet dosage form 90 having two active segments 91 and 93 (shaded) and a middle, inactive segment 92, substantially as shown in FIG. 8, but wherein the tablet includes a score 94 formed in middle segment 92.

FIGS. 10 and 11 depict substantially bi-layer tablets wherein each have an inactive segment for breaking through the tablet. In FIG. 10, tablet dosage form 100 comprises inactive segment 101 forming a substrate segment contiguous with, and providing support for, active unitary segments 102 and 103 (shaded) comprising torsemide as an API, preferably in amounts or about 2 mg or less. Score 104 is formed completely through the layer or layers forming segment 102 and 104 being formed into inactive substrate segment 101. FIG. 11 depicts a segmented tablet dosage form 110 comprising torsemide in active segment 111 (shaded). Active segment 111 comprises a deep score 113 which is formed substantially through, but not completely through the segment, and does not reach into the contiguous inactive segment 112.

The scoring patterns can be provided in the subject dosage forms using conventional scoring procedures, or can be provided using techniques or procedures as described in the published Int'l Application WO 2006/078745, which is incorporated herein by reference.

In use, novel tablets of the subject invention can be broken to provide a portion of the whole tablet wherein the tablet portion comprises a lower dose of torsemide than would be available from a whole, intact tablet. This capability provides an advantage to a physician and a patient of being able to provide a lower (e.g., one half or one quarter tablet) initial dose of torsemide than could be provided by administering a whole tablet or having to provide separate prescription is for different strength tablets in order to flexibly administer varying doses. In addition, a higher initial dose (one whole tablet plus a portion of a broken or divided tablet) can also be provided to effect a desired response in accordance with a physician instruction. Moreover, dosing flexibility or adjustability during a treatment plan can be achieved by providing the subject tablets that are divisible by a patient or caregiver in situations where too high a dose may cause undesired side effects, or when dosage needs to be increased to effect a desired response, such as more rapid onset of effect or to facilitate getting a patient to a goal endpoint. Intermediate dosage strengths can also be an advantage of the subject novelly scored, divisible tablets.

Preferably, the subject methods include use of a torsemide tablet having a score pattern selected from a bisecting, trisecting, quadrisecting or pentasecting score. More preferably, a tablet of the subject invention comprises a score pattern or a tablet configuration that enables the tablet to be easily broken into predictably accurate sub-doses. Such tablet configurations or score patterns are known and exemplified as the tablet configurations shown in FIGS. 8-11. A bisected tablet of FIG. 10, for example, comprising 10 mg of torsemide, would be broken into two tablet portions, or tablettes, which each predictably and accurately contain substantially 5 mg of torsemide. Advantageously, each tablet portion is bioequivalent to one another, provides substantially equivalent drug release rates in vivo and in vitro, and meets composition and content uniformity requirements as promulgated in standard governmental or regulatory guidelines.

More preferably, a scored dosage form in accordance with the subject invention comprises about 2 mg or less of torsemide. Accordingly, a treatment plan for a patient can be initiated by providing a scored (e.g., bisected) 2 mg tablet, administering a half-tablet for seven days, then a whole tablet for seven days, followed by one and one-half tablets for seven days, maintaining the patient on the 3 mg (1.5 tablet) dose indefinitely as needed. Alternatively, providing a quadrisected 10 mg tablet can enable a dosing regimen of 2.5 mg (one-quarter tablet) for the initial dose period (e.g., one week), then increasing the dose to 5 mg (one-half tablet) for the desired period, and the either increasing the dose to 7.5 mg (three-quarters of a tablet) or 10 mg (whole tablet) as needed or otherwise determined by the prescribing physician.

1. A pharmaceutical dosage form comprising about 2 mg or less of torsemide.

2. The pharmaceutical dosage form as in claim 1, wherein said dosage form is tablet.

3. The pharmaceutical dosage form of claim 2 wherein said tablet is a layered tablet.

4. The pharmaceutical dosage form of claim 3 wherein said layered tablet comprises a plurality of segments.

5. The pharmaceutical dosage form as in claim 2, wherein said tablet comprises at least one score.

6. The pharmaceutical dosage form as in claim 1, wherein said dosage form comprises about 2 mg of torsemide.

7. The pharmaceutical dosage form as in claim 1 that comprises about 1 mg of torsemide.

8. The pharmaceutical dosage form as it claim 1, said dosage form further comprising a second antihypertensive agent.

9. The pharmaceutical dosage form as in claim 1, said dosage form further comprising a second cardiovascular agent in addition to torsemide.

10. The pharmaceutical dosage form of claim 1 wherein said dosage form further comprises an anti-diabetic agent.

11. The pharmaceutical dosage form of claim 8 wherein said anti-diabetic agent is a thiazolidinedione.

12. The pharmaceutical dosage form of claim 9 wherein said thiazolidinedione is selected from the group consisting of pioglitazone and rosiglitazone.

13. A pharmaceutical tablet comprising torsemide, said tablet comprising at least two score lines for indicating divisibility of a tablet into at least three portions.

14. The pharmaceutical tablet as in claim 13, wherein said tablet has two scores that delineate three sections of the tablet, wherein each delineated tablet section contains about ⅓ of the total dose of torsemide in the tablet.

15. The pharmaceutical tablet as in claim 13, wherein said tablet has two scores that intersect and delineate four sections of the tablet, wherein each delineated tablet section contains about ¼ of the total dose of torsemide in the tablet.

16. The pharmaceutical tablet as in claim 15 in which the two scores intersect each other at right angles.

17. The pharmaceutical tablet as in claim 13, wherein said tablet comprises three scores.

18. The pharmaceutical tablet of claim 17 in which the three scores are formed parallel to one another to delineate four sections of the tablet, each of the delineated tablet sections containing about ¼ of the total quantity of torsemide in the tablet.

19. The pharmaceutical tablet as in claim 18 wherein said tablet comprises a substantially pharmaceutically inactive segment.

20. The pharmaceutical tablet as in claim 19 in which said inactive segment is scored.

21. The pharmaceutical tablet as in claim 19 in which the tablet comprises two or more unitary segments, each segment comprising an amount of torsemide substantially equal to an amount of torsemide in another unitary segment, each unitary segment being contiguous with the inactive segment.

22. The pharmaceutical tablet as in claim 21 in which the tablet containing a dose of torsemide selected from the group consisting of 2 mg, 3 mg, 4 mg and 6 mg.

23. A method for treating a diabetic or pre-diabetic patient, said method comprising the steps of:
administering to the patient a pharmaceutically acceptable preparation comprising torsemide, wherein said torsemide does n-tot worsen blood sugar levels or glycemic status.

24. The method of claim 23, wherein said administration of the preparation comprising torsemide does not induce diabetes.

25. A preparation as in claim 24, wherein said administration of a preparation comprising torsemide does not worsen blood sugar levels or glycemic status.

26. The method of claim 23 wherein the preparation comprising torsemide provides a dose of about 2 mg or less of torsemide.

27. A pharmaceutically acceptable preparation comprising torsemide as the only active pharmaceutical ingredient that lowers blood sugar or improves glucose tolerance or improves insulin sensitivity when given to a patient with an affliction selected from the group consisting of hyperglycemia, diabetes, pre-diabetes, impaired glucose tolerance, and impaired insulin sensitivity.

28. A pharmaceutically acceptable preparation comprising torsemide and a second active pharmaceutical ingredient in which torsemide independently contributes to improvement of glycemic status in a patient in need of such.

29. A method of treatment of a patient suffering from hypertension, said method comprising:
a. providing a dosage form comprising torsemide; and
b. administering the dosage form to the patient to provide said patient with a dose of less than 2 mg/day of torsemide.

30. The method of claim 29 wherein said dose administration is once daily.

31. The method of claim 29 wherein said dose is 1 mg per day.

32. The method of claim 31 wherein said dose administration is once daily.

33. The method of claim 29 wherein said dosage forms comprises at least one other active ingredient.

34. The method of claim 29 wherein said dosage form is scored.

35. The method of claim 29 wherein said torsemide dose is administered as a portion or fraction of a whole tablet.
